(19) United States
(12) Patent Application Publication
(10) Pub. No.: US 2005/0019848 A1
(43) Pub. Date: Jan. 27, 2005

(54) Blood sugar tester and data uploading method

(76) Inventors: Min-Hwa Lee, Seoul (KR); Moon-Soo Kim, Seoul (KR)

Correspondence Address: MORGAN & FINNEGAN, L.L.P., 3 WORLD FINANCIAL CENTER, NEW YORK, NY, 10281-2101, US

(21) Appl. No.: 10/479,677

(22) PCT Filed: May 14, 2003
(86) PCT No.: PCT/KR03/00949

(30) Foreign Application Priority Data
May 14, 2002 (KR) 10-2002-0026511

(51) Int. Cl. C12Q 1/54

(52) U.S. Cl. 435/14

(57) ABSTRACT

The present invention relates to a blood sugar test device and a method for transmitting a measured blood sugar level to a blood sugar level administration server on the Internet. In one embodiment, blood sugar test function is integrated in a mobile terminal. In another embodiment, a blood sugar test adaptor is connected to a connection terminal of a mobile terminal, the adaptor being equipped with minimal functions to perform blood sugar tests. In addition, both the mobile terminal with integrated blood sugar test function and the mobile terminal connected to the above blood sugar test adaptor can transmit measured blood sugar level to the blood sugar level administration server. A strip case is provided for at an outer surface of the mobile terminal itself in the former, while a strip case is located at an outer surface of the adaptor in the latter.

### TECHNICAL FIELD OF THE INVENTION

The present invention relates to a blood sugar test device and a method for transmitting a measured blood sugar level to a blood sugar level administration server on the Internet, in particular, to a device enabling a mobile communication terminal to test blood sugar level by integrating blood sugar test function in the communication terminal and a device enabling a mobile communication terminal to display blood sugar level measured by a blood sugar test adaptor connected to a connection terminal of the mobile communication terminal, the blood sugar test adaptor being equipped with minimal functions to perform blood sugar tests. In addition, both a mobile communication terminal with integrated blood sugar test function and a mobile communication terminal connected to the above blood sugar test adaptor can transmit measured blood sugar level to blood sugar level administration server on the Internet.

### BACKGROUND ART

A blood sugar test, being measurement of glucose level in blood, shall be conducted everyday to a patient with fluctuating glucose level. Repeated blood sugar test even within one single day shall be made for a patient diagnosed as a heavy diabetic or other specific disease. Although a blood sugar test is frequently conducted in a clinic or other medical institution, various portable blood sugar test devices are currently in supply to enable a patient to test his glucose level personally in his everyday life. For a self-test of glucose level, a patient normally requires a blood sugar test device capable of measuring a glucose level, a needle (lancet) for taking blood sample, a strip which is a test paper, and a diabetic diary for recording the test results.

FIG. 1 shows a conventional portable blood sugar test device 100. This blood sugar test device functions as follows: If a test strip 130 is applied with a blood sample and inserted into connector 120 of the blood sugar test device, glucose level of the blood sample is automatically measured and the measured glucose level is displayed on the display window 110.

A glucose level thus measured and recorded in a diabetic diary can serve as a guideline for controlling and monitoring glucose fluctuation by diet, exercise, stress, etc. as well as for administration of a diabetic. A well administration of glucose level can prevent complications in the eyes, the kidneys, the nerval systems, the blood vessels, etc. caused by high glucose level, and contribute to a rapid monitoring of a high or low blood pressure as well as curing thereof.

However, for self-test of glucose level, a patient must carry a blood sugar test device, a strip case, and a diabetic diary for recording the test results, which is very inconvenient in daily life. After a test, he shall record the test result and take the records to a physician for further diagnostic and treatment.

### DISCLOSURE OF THE INVENTION

The present invention, conceived to solve the above problems, aims to provide a blood test device allowing a mobile communication terminal or a blood sugar test adaptor to test a glucose level, whereby a strip case is provided for at an out surface of the mobile communication terminal or of the blood sugar test adaptor, thus freeing a user from carry a strip case additionally.

Furthermore, the present invention aims to ease diagnosis and treatment of a diabetic by a physician by allowing the physician to check glucose level of a patient in real time through automatically recording results of blood sugar tests at a blood sugar level administration server on the Internet, thus freeing a user from the necessity of recording results of blood sugar tests.

A still further objective of the present invention is to provide a blood sugar test adaptor that allows a conventional mobile communication terminal to be used as a blood sugar test device by displaying a measured glucose level on the display window of the mobile communication terminal, not like a conventional blood sugar test device, and thus, is convenient in carrying and competitive in both in size and price in comparison to a conventional portable blood sugar test device.

### BRIEF DESCRIPTION OF THE DRAWINGS

FIG. 1 is a plane view showing a conventional portable blood sugar test device.

FIG. 2 shows an internal block diagram of a mobile communication terminal with integrated blood sugar test function.

FIG. 3 is a perspective view of a mobile communication terminal with integrated blood sugar test function.

FIG. 4 is a perspective view showing how a mobile communication terminal is connected to a blood sugar test adaptor.

FIG. 5 shows an internal block diagram of a blood sugar test adaptor.

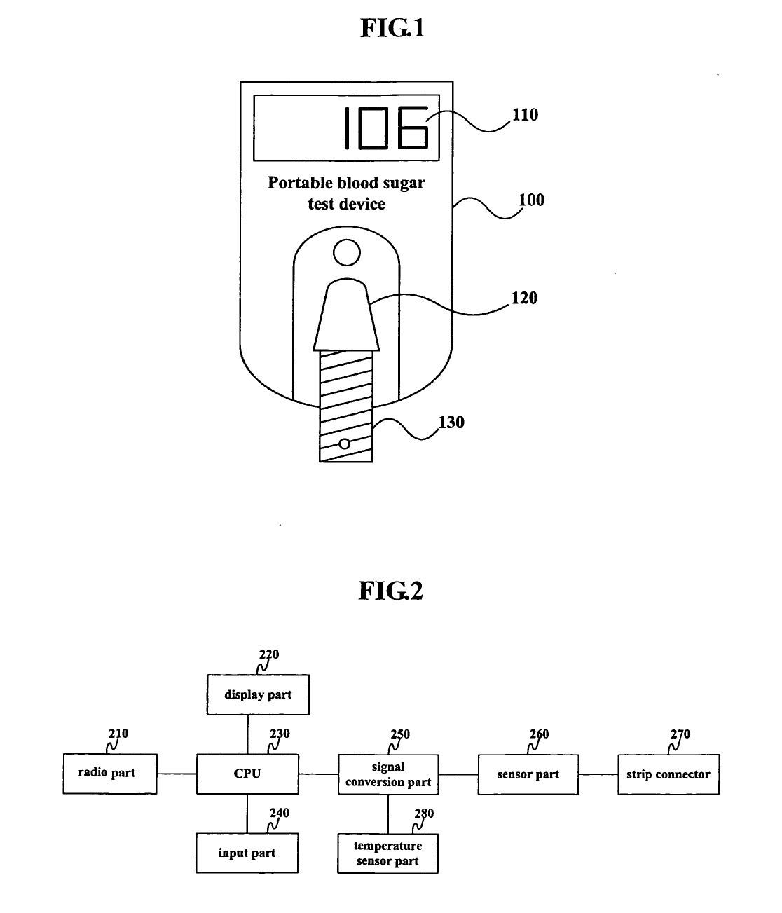

### DESCRIPTION OF THE MAIN COMPONENTS IN DRAWINGS

100: portable blood sugar test device

300: mobile communication terminal with integrated blood sugar test function

500: blood sugar test adaptor

330, 570: strip

### BEST MODE FOR CARRYING OUT THE INVENTION

In order to achieve the above objectives, the present invention provides two types of blood sugar test device capable of measuring glucose level using a mobile communication terminal.

One of the above devices relates to a mobile communication terminal with blood sugar test function integrated in it (to be called hereinafter, “mobile communication terminal with integrated glucose test function”) and the other device relates to an adaptor equipped with minimal blood sugar test function (to be called hereinafter, “glucose test adaptor”), which is connected to a serial port of a mobile communication terminal and displays a glucose level measured by the glucose test adaptor on a display of the mobile communication terminal connected thereto. Both the above mobile communication terminal with integrated glucose test function and a mobile communication terminal connected to the above glucose test adaptor are capable of transmitting glucose level measured by them to a blood sugar administration server on the Internet, whereby a strip case is provided for at an outer surface of the mobile communication terminal with integrated glucose test function in the former, while a strip case is provided for at an outer surface of the glucose test adaptor in the latter.

A mobile communication terminal with integrated glucose test function in accordance with the present invention comprises a central processing part, a radio part, an input part, a strip connector allowing a strip applied with blood sample to be inserted into it, a sensor part for measuring electric current corresponding to a glucose level, a temperature sensor part for measuring temperature, a signal conversion part for converting a measured electric current into a glucose value referring to the respective temperature, and a display part for displaying a measured glucose level on display window of the mobile communication terminal.

A glucose test adaptor in accordance with the present invention comprises corresponding parts to a strip connector, a sensor part, a temperature sensor part, and a signal conversion part in the mobile communication terminal, in addition to a central processing part for controlling function parts of the glucose test adaptor and a serial port for transmitting measured glucose level to the mobile communication terminal.

In the following, a detailed description of the preferred embodiments of the present invention is given making reference to the accompanying drawings.

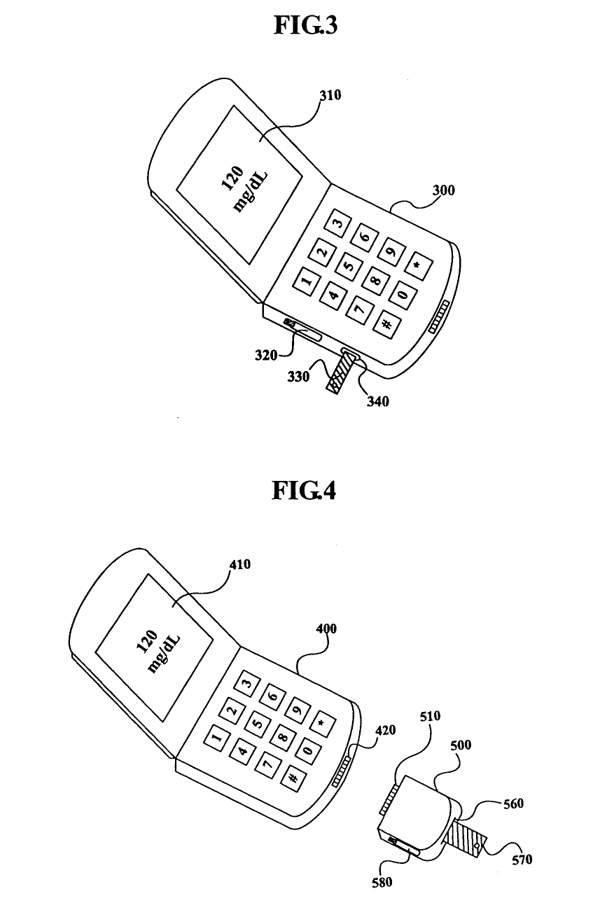

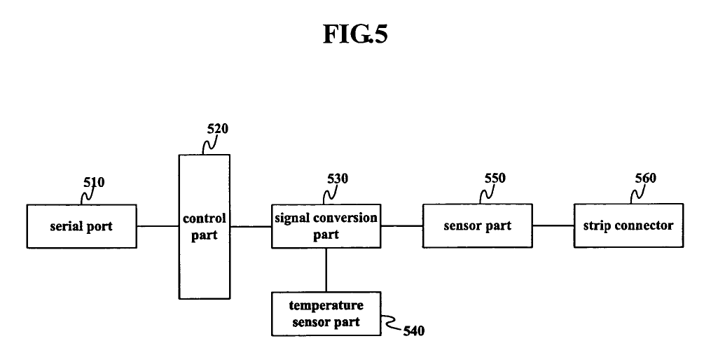

FIGS. 2 and 3 relate to a mobile communication terminal with integrated glucose test function, while FIGS. 4 and 5 relate to a glucose test adaptor.

FIG. 2 shows an internal block diagram of a mobile communication terminal with integrated glucose test function.

The strip connector 270 is a part into which a strip applied with a blood sample shall be inserted for measuring the amount of glucose contained in the blood sample. A strip is a test paper for measuring glucose level in a manner that a patient takes a blood sample using a needle or lancet, applies the blood sample on the strip and then inserts the strip into the strip connector.

The sensor part 260 is a part for measuring electric current present in the blood sample. Upon insertion of a strip into the strip connector 270 the sensor 260 is automatically activated to measure the electric current, and then, to transmit the measured value to the signal conversion part 250. Since this sensing occurs automatically with insertion of a strip into the strip connector without manipulation by a user, a separate user interface is not required.

The sensing is performed on the same principle as in a conventional portable blood sugar test device, i.e. by measuring electric current flowing through the sensor when electrons are emitted during oxidation process of glucose through glucose oxidation enzyme, and by computing a corresponding glucose level. Accordingly, the sensor part 260 measures electric current during glucose oxidation process in blood and then transmits the measured value to the signal conversion part 250, whereupon the latter converts the electric current value into a corresponding glucose value.

The temperature sensor part 280 measures the temperature at the time of measuring a glucose level and transmits the measured result to the signal conversion part 250 so that an eventual error in the glucose value through temperature changes can be corrected.

The signal conversion part 250 converts the current value received from the sensor part 260 into a glucose value, transmits the same to the central processing part 230, and corrects an error in the measured glucose value based on the temperature transmitted from the temperature sensor part 280.

The central processing part 230 controls not only all communication functions of the mobile communication terminal, but also all functional parts relating to the glucose level measuring. It further functions to transmit a measured glucose value to the display part 220 so that a glucose level is displayed on the display window of the mobile communication terminal to allow a user to view the displayed glucose level.

The display part 220 enables a user to view a glucose value measured by displaying the same.

The input part 240 provides a user interface for communication, which is the principal function of a mobile communication terminal.

FIG. 3 is a perspective view of a mobile communication terminal with integrated blood sugar test function as a strip applied with blood sample is inserted into it.

Here, result of a glucose test is displayed on the display window 310 of the mobile communication terminal, an example of such glucose value (120 mg/dl) is shown in FIG. 3.

As strips of different size are used for diverse conventional portable blood sugar test devices, a strip 300 having an appropriate size for a strip connector 340 is used in a mobile communication terminal with integrated glucose test function in accordance with the present invention. Although the strip connector 340 is provided for at one side of the mobile communication terminal in the above drawing, it can be provided for at other locations as well, e.g. at upper or bottom part of the mobile communication terminal.

Further, the mobile communication terminal can also comprise a strip case 320 for storing strips. Although the strip case 320 is installed at the right side of the mobile communication terminal in the above drawing, it can be located at other available locations of the mobile communication terminal as well, such as at the left side, at the bottom part, at the rear side with battery case, etc.

FIG. 4 is a perspective view showing how a mobile communication terminal is connected to an external blood sugar test adaptor.

Upon insertion of a strip 570 applied with blood sample into the strip connector 560, the glucose test adaptor 500 measures the glucose level and transmits the measured glucose value to the mobile communication terminal 400 via a serial port 510 of the glucose test adaptor. Upon receiving a glucose value, the mobile communication terminal 400 displays the same on its display window 410 and at the same time transmits the glucose value to a glucose level administration server on the Internet so that the measured glucose value can be stored in the server.

Strips 570 in use for the conventional portable blood sugar test devices are also applicable in the present invention. Further, the glucose test adaptor 500 may comprise a strip case 580 for storing strips. Although the strip case 580 is installed at a side of the glucose test adaptor in the above drawing, it may be located at other available locations of the glucose test adaptor as well, such as at the front side, at the rear side, etc.

However, a mobile communication terminal to be connected to the glucose test adaptor shall comprise programs for a glucose test adaptor interface as well as for automatic reporting of glucose values to a glucose level administration server. Such programs may be loaded to a mobile communication terminal either through a manufacturing process, or through downloading of such programs from a service center of mobile phone manufacturer.

FIG. 5 shows an internal block diagram of a glucose test adaptor.

Since the strip connector 560, the sensor part 550, the temperature sensor part 540, and the signal conversion part 530 have the same functions as the corresponding parts in the mobile communication terminal with integrated glucose test function in FIG. 2, an explanation thereon is omitted here. Accordingly, a description on the remaining parts is given below.

The control part 520, being a part for controlling all function parts of the glucose test adaptor, transmits glucose level data to the serial port for further transmission to the mobile communication terminal.

The serial port 510, being a part for RS-232 serial communication with the mobile communication terminal, may also be connected to the mobile communication terminal via a cable. However, in present embodiment example of the present invention, the glucose test adaptor is constructed to connect directly to the mobile communication terminal by insertion thereof into the serial port of the mobile communication terminal.

Both a mobile communication terminal with integrated glucose test function as in FIG. 3 and a mobile communication terminal connected to a glucose test adaptor as in FIG. 4 transmit measured glucose values to a glucose level administration server on the Internet after completion of such measuring. For this purpose, the mobile communication terminal automatically connects to the glucose level administration server upon measuring a glucose value and logins with a user ID. After successful login, the mobile communication terminal transmits the measured glucose value to the glucose level administration server, whereupon the latter stores the transmitted glucose value in an appropriate area of database for the respective individual user. This automatic storage in the glucose level administration server frees a user from the troublesome recording of every result of glucose tests in a diabetic diary, and thus, provides convenience to the user. Moreover, since a diabetic requires periodic checking and/or treatment by a physician, the present invention provides great advantage both for the patient and the physician by allowing the physician to view a measured glucose value of a remote patient stored in the glucose level administration server in real time.

Although the present invention has been described above with reference to the embodiment examples and the accompanying drawings, the scope of rights of the present invention is not limited thereto, but rather, shall be determined by the claims attached herein after and their equivalents, allowing various modifications and adaptations without departing the spirit of the present invention, as those skilled in the art will understand.

### INDUSTRIAL APPLICABILITY

As described above, the present invention, by allowing a user to test glucose level using a mobile communication terminal including a strip case, frees the user from troublesome carrying of a conventional blood sugar tester and a separate strip case as it was necessary for a conventional blood sugar test device. The present invention further frees a user from annoying recording of the results of glucose tests in a diabetic diary without the necessity to carry a diabetic diary with him, by directly transmitting results of glucose tests from a mobile communication terminal to a glucose level administration server on the Internet, and thus, enables a remote physician to view the measured glucose values in real time.

1. A mobile communication terminal with integrated glucose test function comprising:
a strip connector into which a strip applied with blood sample can be inserted,
a sensor part for measuring electric current existent in blood sample on said strip,
a temperature sensor part for measuring temperature at the time of a glucose test,
a signal conversion part for converting said measured electric current transmitted from said sensor part into a glucose value referring to said temperature transmitted from said temperature sensor part,
a radio part that transmits said glucose value measured to a glucose level administration server on the Internet through radio communication with a mobile communication base station,
a display part for displaying measured glucose value,
an input part for user interface, and
a central processing part which controls function parts of said mobile communication terminal and transmits said glucose value received from said signal conversion part to said display part as well as to said radio part.

2. The mobile communication with integrated glucose test function as set forth in claim 1, further comprising a strip case for storage of strips at an outer surface thereof, said strips being strips to be applied with blood sample.

3. A glucose test adaptor for use in connection with a mobile communication terminal comprising:
a strip connector into which a strip applied with blood sample can be inserted,
a sensor part for measuring electric current existent in blood sample on said strip,
a temperature sensor part for measuring temperature at the time of a glucose test,
a signal conversion part for converting said measured electric current transmitted from said sensor part into a glucose value referring to said temperature transmitted from said temperature sensor part,
a serial port for serial communication with a mobile communication terminal capable of transmitting measured glucose value to said mobile communication terminal, and
a central processing part which controls function parts of said glucose test adaptor and transmits said glucose value received from said signal conversion part to said serial port.

4. The glucose test adaptor for use in connection with a mobile communication terminal as set forth in claim 3, comprising a strip case for storage of strips at an outer surface thereof, said strips being strips to be applied with blood sample.

5. A method for transmitting measured glucose value to a glucose level administration server on the Internet via a radio network, comprising the steps of:
automatic connecting to and logging in a predetermined glucose level administration server on the Internet by a mobile communication terminal after a glucose test has been performed by said mobile communication terminal with integrated glucose test function or by a glucose test adaptor connected to said mobile communication terminal, and
transmitting measured glucose value to a glucose level administration server on the Internet by said mobile communication terminal via a radio network.
